(19) United States
(12) Patent Application Publication
(10) Pub. No.: US 2008/0195210 A1
(43) Pub. Date: Aug. 14, 2008

(54) Intervertebral Disc Restoration

(75) Inventors: Zoran Milijasevic, New South Wales (AU); Ashish Diwan, New South Wales (AU); Richard Charles Appleyard, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: COLUMNA PTY LTD, Kogarah, New South Wales (AU)

(21) Appl. No.: 11/817,586

(22) PCT Filed: Mar. 1, 2006
(86) PCT No.: PCT/AU2006/000267

§ 371 Date: Feb. 28, 2008

(30) Foreign Application Priority Data
Mar. 1, 2005 (AU) 2005900952

(51) Int. Cl. A61F 2/44

(52) U.S. Cl. 623/17.16; 623/17.12

(57) ABSTRACT

An intervertebral disc implant (10) includes an envelope (12) of a stretchable and elastically deformable elastomeric material. The envelope includes an attaching formation (74) for attachment of an introducer (76) to enable the envelope, in a collapsed state, to be introduced into a volume of an intervertebral disc that has undergone a nucleotomy. A filler material (14) is receivable in the envelope via the introducer to cause the envelope to expand elastically to conform substantially to the volume in which the envelope is received.

### CROSS-REFERENCE TO RELATED APPLICATIONS

The present application claims priority from Australian Provisional Patent Application No. 2005900952 filed on 1 Mar. 2005, the contents of which are incorporated herein by reference.

### FIELD OF THE INVENTION

This invention relates to intervertebral disc restoration. More particularly, the invention relates to an intervertebral disc implant, to a system for, and a method of, implanting an intervertebral disc implant and to an introducer for the system.

### BACKGROUND TO THE INVENTION

Joints of the musculoskeletal system of the human or animal body rely on the presence of healthy cartilaginous tissue for proper operation. Cartilaginous tissue can degenerate due to a number of causes, eg. age or injury. Degradation of the tissue can reach a point where movement can cause severe discomfort and pain.

In the case of the spinal column, it comprises a series of 26 mobile vertebral bones, or vertebrae, connected by 75 stable articulations that control motion. The vertebrae are generally divided into posterior and anterior elements by thick pillows of bone called pedicles. The anterior element of the vertebra is a kidney shaped prism of bone with a concavity directed posteriorly and has flat superior and inferior surfaces called end plates. An intervertebral disc is sandwiched between adjacent pairs of vertebrae forming a joint between the adjacent pair of vertebrae. These discs are viscoelastic structures comprising a layer of strong, deformable, soft tissue. The intervertebral discs are subjected to a considerable variety of forces and moments resulting from the movements and loads of the spinal column. Each intervertebral disc has two components, being the annulus fibrosis surrounding a nucleus pulposus. The intervertebral disc cooperates with end plates of the vertebrae between which it is sandwiched.

The primary function of the nucleus pulposus of the disc is to give the disc its elasticity and compressibility characteristics to assist in sustaining and transmitting weight. The annulus fibrosis contains and limits the expansion of the nucleus pulposus during compression and also holds together successive vertebrae, resisting tension and torsion in the spine. The end plates of the vertebrae are responsible for the influx of nutrients into the disc and the efflux of waste products from within the disc.

With age or injury, a degenerative process of the disc may occur whereby its structures undergo morphological and biological changes affecting the efficiency with which the disc operates. Thus, the nucleus pulposus may reduce in volume and dehydrate resulting in a load reduction on the nucleus pulposus, a loss in intradiscal pressure and, hence, additional loading on the annulus fibrosis. In a normally functioning disc, the intradiscal pressure generated results in deformation of the end plates of the adjacent vertebrae generating the natural pumping action which assists in the influx of the nutrients and the efflux of waste products as stated above. A drop in intradiscal pressure therefore results in less end plate deformation. The nutrients supplied to the discal tissue is reduced and metabolic wastes are not removed with the same efficiency. This contributes to a degenerative cascade.

Radial and circumferential tears, cracks and fissures may begin to appear within the annulus fibrosis. If these defects do not heal, some of the nuclear material may begin to migrate into the defects in the annulus fibrosis. Migration of the nuclear material into the annulus fibrosis may cause stretching and delamination of layers of the annulus fibrosis resulting in back pain due to stimulation of the sinu-vertebral nerve. An intervertebral disc without a competent nucleus is unable to function properly. Further, since the spine is a cooperative system of elements, altering the structure and mechanics at one location of the spinal column may significantly increase stresses experienced at adjacent locations thereby further contributing to the degenerative cascade.

In the past, operative intervention has occurred to relieve lower back pain arising from intervertebral disc degeneration. Most of this operative intervention has been by way of a discectomy where leaking nuclear material is removed or, alternatively, fusion. The primary purpose of a discectomy is to excise any disc material that is impinging on the spinal nerve causing pain or sensory changes. Fusion means eliminating a motion segment between two vertebrae by use of a bone graft and sometimes internal fixation. Biomechanical studies show that fusion alters the biomechanics of the spine and causes increased stresses to be experienced at the junction between the fused and unfused segments. This promotes degeneration and begins the degenerative cycle anew. Clearly, being an invasive operative procedure, fusion is a risky procedure with no guarantee of success.

Due to the minimal success rate of the previous two procedures, as well as their inability to restore complete function to the spinal column, alternative treatments have been sought in the form of artificial disc replacements. Theoretical advantages of artificial disc replacement over a fusion procedure include preservation or restoration of segmental motion in the spine, restoration of intervertebral architecture and foraminal height, sparing of adjacent segments of the spine from abnormal stresses and restoration of normal biomechanics across the lumbar spine. The established artificial disc replacement procedure consists of techniques that require a surgical incision on the abdomen, retraction of large blood vessels, a total excision of the anterior longitudinal ligament, anterior and posterior annulus along with the nucleus and near total removal of the lateral annulus and implantation of an articulated prosthesis. This is a major spinal column reconstruction operation carried out by a very invasive technique.

There is therefore a need for a surgical procedure which, as far as possible, restores the biomechanics of joints such as those between adjacent vertebrae of the spine by the provision of a tissue prosthesis mimicking natural, healthy cartilaginous tissue as well as a means of carrying out the surgical procedure in a minimally invasive manner.

### SUMMARY OF THE INVENTION

According to a first aspect of the invention, there is provided an intervertebral disc implant which includes

an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and

a filler material receivable in the envelope via the introducer to cause the envelope to expand elastically to conform substantially to the volume in which the envelope is received, in use.

By “elastically deformable” is meant that the envelope is able to expand without plastic deformation at least in its normal operating range, i.e. the maximum size to which the envelope would need to expand to conform as completely as possible to the volume. Further, “volume” is to be understood as the space or void remaining in the disc after the nucleotomy has been performed.

Preferably, the envelope is of a silicone material.

The attaching formation may comprise a filler tube mountable to the introducer, the attaching formation including a closure device to inhibit back flow of filler material. Any suitable closure device may be employed such as, for example, a one-way, or non-return, valve, a filler tube extending outwardly from the remainder of the envelope to be closed off in a suitable manner or a filler tube extending into the interior of the envelope and which is crimped closed by the surrounding filler material upon withdrawal of the introducer.

In one embodiment, the filler material may comprise a plurality of discrete, biocompatible elements. The elements may include, singly or in combination, beads, elongate elements and expansible elements. The elements may be biocompatible plastics, biocompatible metals, biocompatible ceramics, organic or biological elements, or a combination of the foregoing. Further, the elements may be provided in a mixture of sizes.

The elongate elements may be selected from the group consisting of fibres, lengths of filamentary elements such as lengths of string, bristle carrying elements such as bottle brush-like elements, and helical elements such as lengths of coiled wires.

The discrete elements may be arranged in suspension in a filler within the volume. Preferably, the filler is an elastomeric, curable filler.

Each expansible element may be configured to change from a first configuration for insertion into the envelope to a second configuration which causes the envelope to conform substantially to the volume. Further, each expansible element may be configured to be received, in its first configuration, in the introducer for introduction into the envelope.

Each expansible element may, in its rest condition, adopt its second configuration. Further, each expansible element may include a biocompatible, shape memory alloy, such as nitinol, which causes the element to adopt its second configuration in the envelope after ejection from the introducer.

In another embodiment, the filler material may be a foamed material which is introduced in a compressed state via the introducer into the interior of the envelope where it expands to its relaxed state to cause the envelope to conform to the volume. The foamed material may be a polymeric material such as a polyethylene.

In yet a further embodiment, the filler material may comprise a plurality of discrete bands of a resiliently flexible material. The bands may be configured to be arranged concentrically within the envelope. The bands may have a height approximating that of the volume.

The envelope may carry at least one layer of a tissue ingrowth material. The layer may be a polyester material such as Dacron (Registered Trade Mark).

According to a second aspect of the invention, there is provided an intervertebral disc implant which includes

an envelope, the envelope including an attaching formation for attachment to an introducer to enable the envelope, in a collapsed state, to be introduced into a volume of an intervertebral disc that has undergone a nucleotomy; and

a filler material receivable in the envelope after placement of the envelope in the volume of the disc, in use, to cause expansion of the envelope to conform to the volume, the filler material comprising a plurality of discrete, elongate elements introducible, via the introducer, into an interior of the envelope.

The envelope may be of an expansible material such as an elastomeric material having an elongation of at least 100% and, preferably, up to about 1000%, for example, silicone.

The envelope may carry at least one layer of a tissue ingrowth material.

Further, the envelope may define a filler opening and may include a closure element for closing the opening after introduction of the filler material.

In one embodiment the elongate element may be selected from the group consisting of fibres, lengths of filamentary elements, bristle carrying elements and helical elements.

The elongate elements may be arranged in suspension in a filler within the volume. Preferably, the filler is an elastomeric, curable filler.

In another embodiment, the elongate elements may comprise a plurality of discrete bands of a resiliently flexible material. The bands may be configured to be arranged concentrically within the envelope.

In a further embodiment, the elongate elements may be expansible elements. Each expansible element may be configured to change from a first configuration for insertion into the envelope to a second configuration which causes the envelope to conform substantially to the volume. Each expansible element may be configured to be received, in its first configuration, in the introducer for introduction into the envelope. Further, each expansible element may, in its rest condition, adopt its second configuration.

According to a third aspect of the invention, there is provided an intervertebral disc implant which includes

an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and

a filler material receivable in the envelope via the introducer to cause the envelope to expand elastically to conform substantially to the volume in which the envelope is received, in use, the filler material being a foamed material which is introduced in a compressed state via the introducer into the interior of the envelope where it expands to its relaxed state to cause the envelope to conform to the volume.

The foamed material may be a polymeric material.

The envelope may carry at least one layer of a tissue ingrowth material.

The envelope may define a filler opening and may include a closure element for closing the opening after introduction of the packing material.

According to a fourth aspect of the invention, there is provided an intervertebral disc implant which includes

an envelope, the envelope including an attaching formation for attachment to an introducer to enable the envelope, in a collapsed state, to be introduced, in a minimally invasive manner, into a volume of an intervertebral disc that has undergone a nucleotomy; and

a filler material receivable in the envelope after placement of the envelope in the volume of the disc, in use, to cause expansion of the envelope to conform to the volume, the filler material comprising, in combination, a curable filler material and a plurality of discrete, biocompatible elements contained, in use, in the filler material within the envelope.

The envelope may be of an expansible material.

The envelope may carry at least one layer of a tissue ingrowth material.

The envelope may define a filler opening and may include a closure element for closing the opening after introduction of the packing material.

The elements may include, singly or in combination, beads, elongate elements and expansible elements. The elongate elements may be selected from the group consisting of fibres, lengths of filamentary elements, bristle carrying elements and helical elements.

Each expansible element may be configured to change from a first configuration for insertion into the envelope to a second configuration which causes the envelope to conform substantially to the volume. Further, each expansible element may be configured to be received, in its first configuration, in the introducer for introduction into the envelope. Each expansible element may, in its rest condition, adopt its second configuration.

The filler may be an elastomeric, curable filler.

According to a fifth aspect of the invention, there is provided an intervertebral disc implant which includes

an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and

a filler material receivable in the envelope via the introducer to cause the envelope to expand elastically to conform substantially to the volume in which the envelope is received, in use, the filler material being an elastomeric material having a viscosity of at least 500000 cP.

Preferably, the elastomeric material is a silicone.

The envelope may carry at least one layer of a tissue ingrowth material.

The envelope may define a filler opening and may include a closure element for closing the opening after introduction of the packing material.

According to a sixth aspect of the invention, there is provided an intervertebral disc implant which includes

an envelope receivable in a volume of an intervertebral disc that has undergone a nucleotomy, the envelope defining a plurality of chambers, the chambers being configured so that, when at least certain of the chambers contain a filler material, the envelope conforms substantially to the volume of the disc;

a filler material receivable in the at least certain of the chambers; and

at least one of the chambers having a filler mechanism associated with it.

The chambers may be defined by wall portions of the envelope, wall portions of some of the chambers being of a different wall thickness than wall portions of other chambers. In addition, wall portions of some of the chambers may be of a different material than wall portions of other chambers. Still further, the filler material receivable in at least one of the chambers may differ from the filler material that is receivable in at least one other of the chambers.

The envelope may include an attaching formation for attachment to a tubular introducer to enable the envelope, in a collapsed state, to be introduced, in a minimally invasive manner, into the volume of the disc.

Each chamber in which filler material is receivable may have a filler mechanism associated with it. The filler mechanism may be a one-way device that, upon closure, inhibits back flow of filler material. Preferably, the filler mechanism of an outer chamber of the envelope may be implemented as the attaching formation.

The envelope may carry at least one layer of a tissue ingrowth material.

The invention extends to a system for implanting an intervertebral disc implant, the system comprising

an implant as described above with reference to the sixth aspect of the invention; and

an introducer, the introducer comprising a plurality of filler tubes, each tube communication independently of any other tube with its associated chamber of the envelope for charging filler material into the associated chamber.

According to a seventh aspect of the invention, there is provided an intervertebral disc implant which includes at least one element which changes from a first configuration for insertion into a volume of an intervertebral disc that has undergone a nucleotomy to a second configuration in which the at least one element conforms substantially to the volume, the at least one element being configured to be received, in its first configuration, in an introducer to be inserted into the volume of the disc.

The at least one element, in its first configuration, may be elongate and, in its second configuration, may adopt a shape conforming substantially to the volume. The at least one element may include a biocompatible, shape memory alloy which causes the element to adopt its second configuration in the volume after ejection from the introducer.

Further, in one embodiment, the at least one element, in its relaxed state, may be in the first configuration, the at least one element including a retention device for retaining the at least one element in the second configuration after ejection from the introducer.

In another embodiment, the implant may include

an envelope receivable in a collapsed state in the volume; and

a plurality of the elements receivable in the envelope, the plurality of elements causing the envelope to expand substantially to conform to the volume.

According to an eighth aspect of the invention, there is provided a system for implanting an intervertebral disc implant as claimed in any one of the preceding claims, the system including

an introducer having a proximal and a distal end, a mount for the envelope of the implant being arranged at or adjacent the distal end of the introducer;

a source of filler material connectable to the proximal end of the introducer; and

a displacement mechanism for displacing the filler material along the introducer to be ejected from the introducer into the envelope, in use.

The introducer may comprise at least one tubular member. Instead, the introducer may comprise at least two tubular members arranged in a telescopic fashion, the tubular members being reciprocally displaceable relative to one another.

An innermost one of the tubular members may carry the displacement mechanism. The displacement mechanism may comprise a ratchet arrangement for urging the filler material along the introducer into the envelope.

According to a ninth aspect of the invention, there is provided a method of implanting an intervertebral disc implant into an intervertebral disc, the method including

percutaneously performing a nucleotomy on the disc to remove a nucleus pulposus of the disc to create a volume;

inserting an envelope of the implant into the volume;

charging an interior of the envelope with filler material in a manner to allow the envelope to expand to conform substantially to the volume; and

causing the interior of the envelope to be closed off to retain the filler material within the envelope, the filler material being selected to mimic natural biomechanical characteristics of the nucleus pulposus of the disc.

The method may include inserting the envelope into the volume using an introducer, the envelope being placed in a collapsed state on a distal end of the introducer and inserted percutaneously through an opening in an annulus of the disc. The opening may be the same opening via which the nucleotomy had been performed.

The method may include charging the filler material into the interior of the envelope through the introducer.

Further, the method may include closing off the interior of the envelope by sealing a wall of the envelope. Preferably, the method includes closing off the interior of the envelope by the action of withdrawing the introducer from the envelope.

According to a tenth aspect of the invention, there is provided a method of implanting an intervertebral disc implant into an intervertebral disc, the method including

percutaneously performing a nucleotomy on the disc to remove a nucleus pulposus of the disc to create a volume;

inserting an introducer into an opening formed in an annulus of the disc; and

introducing into the volume, via the introducer, at least one element which changes from a first configuration, in which the at least one element is able to be inserted into the introducer, to a second configuration in which the at least one element conforms substantially to the volume.

The method may include using a single element which, in its second configuration, conforms substantially to the volume of the disc. Instead, the method may include using a plurality of elements which together, when each such element is in its second configuration, conform substantially to the volume of the disc. In the latter case, the method may include, prior to insertion of the elements in the volume, introducing an envelope, in a collapsed state, into the volume and introducing the elements into the envelope to cause the envelope to expand to conform substantially to the volume of the disc.

The method may include, after introduction of the elements into the envelope, closing off a filler opening of the envelope. Preferably, the method includes closing off the filler opening of the envelope by withdrawal of the introducer from the filler opening of the introducer.

According to an eleventh aspect of the invention, there is provided an introducer for introducing an intervertebral disc implant into a disc that has undergone a nucleotomy, the introducer including

at least two sleeves arranged telescopically with respect to each other; and

a displacement mechanism arranged on an operatively inner surface of an innermost one of the sleeves for assisting in displacing filler material along the sleeves into an interior of the disc, in use.

The displacement mechanism may comprise a ratchet arrangement for urging the filler material along the sleeve.

### BRIEF DESCRIPTION OF DRAWINGS

FIGS. 1a, 1b and 1c show, respectively, front, side and plan views of an intervertebral disc implant, in accordance with a first embodiment of the invention, in use;

FIGS. 2a, 2b and 2c show, respectively, front, side and plan views of an intervertebral disc implant, in accordance with a second embodiment of the invention, in use;

FIGS. 3a, 3b and 3c show, respectively, front, side and plan views of an intervertebral disc implant, in accordance with a third embodiment of the invention, in use;

FIGS. 4a, 4b and 4c show, respectively, front, side and plan views of an intervertebral disc implant, in accordance with a fourth embodiment of the invention, in use;

FIGS. 5a, 5b and 5c show, respectively, front, side and plan views of an intervertebral disc implant, in accordance with a fifth embodiment of the invention, in use;

FIGS. 6a, 6b and 6c show, respectively, front, side and plan views of an intervertebral disc implant, in accordance with a sixth embodiment of the invention, in use;

FIGS. 7a, 7b and 7c show, respectively, front, side and plan views of an intervertebral disc implant, in accordance with a seventh embodiment of the invention, in use;

FIGS. 8a, 8b and 8c show, respectively, front, side and plan views of an intervertebral disc implant, in accordance with an eighth embodiment of the invention, in use;

FIGS. 9a, 9b and 9c show, respectively, front, side and plan views of an intervertebral disc implant, in accordance with a ninth embodiment of the invention, in use;

FIGS. 10a, 10b and 10c show, respectively, front, side and plan views of an intervertebral disc implant, in accordance with a tenth embodiment of the invention, in use;

FIGS. 11a, 11b and 11c show, respectively, front, side and plan views of an intervertebral disc implant, in accordance with an eleventh embodiment of the invention, in use.

FIG. 12 shows a schematic side view of an intervertebral disc implant, in accordance with a twelfth embodiment of the invention, in its first configuration;

FIG. 13 shows a schematic plan view of the implant of FIG. 12 in its second configuration;

FIG. 14 shows a schematic side view of an intervertebral disc implant, in accordance with a thirteenth embodiment of the invention, in its first configuration;

FIG. 15 shows a schematic plan view of the implant of FIG. 14 in its second configuration;

FIG. 16 shows a schematic side view of an intervertebral disc implant, in accordance with a fourteenth embodiment of the invention, in its first configuration;

FIG. 17 shows a schematic plan view of the implant of FIG. 16, in use, in its second configuration;

FIG. 18 shows a schematic plan view of an intervertebral disc implant, in accordance with a fifteenth embodiment of the invention;

FIG. 19 shows a schematic plan view of an intervertebral disc implant, in accordance with a sixteenth embodiment of the invention;

FIG. 20 shows a schematic three dimensional view of the implant of FIG. 19;

FIG. 21 shows a schematic three dimensional view of an intervertebral disc implant, in accordance with a seventeenth embodiment of the invention;

FIG. 22 shows a schematic three dimensional view of an intervertebral disc implant, in accordance with an eighteenth embodiment of the invention;

FIG. 23 shows a three dimensional view of an intervertebral disc implant, in accordance with a nineteenth embodiment of the invention;

FIG. 24 shows a sectional side view of the implant of FIG. 23;

FIG. 25 shows a three dimensional view of the implant of FIG. 23, in use;

FIG. 26 shows a schematic three dimensional view of an intervertebral disc implant, in accordance with a twentieth embodiment of the invention;

FIG. 27 shows a schematic, sectional plan view of an intervertebral disc implant, in accordance with a twenty-first embodiment of the invention;

FIG. 28 shows sectional end view taken along line A-A in FIG. 27;

FIG. 29 shows a schematic, sectional three dimensional view of an intervertebral disc implant, in accordance with a twenty-second embodiment of the invention;

FIG. 30 shows, on an enlarged scale, the detail encircled by “A” in FIG. 29;

FIG. 31 shows the detail of FIG. 30 in a collapsed configuration;

FIG. 32 shows a three dimensional view of a system, in accordance with another embodiment of the invention, for implanting an intervertebral disc implant;

FIG. 33 shows, on an enlarged scale, a three dimensional view of the system of FIG. 32; and

FIG. 34 shows a schematic, sectional side view of an introducer for a system, in accordance with a further embodiment of the invention, for implanting an intervertebral disc implant;

### DETAILED DESCRIPTION OF EXEMPLARY EMBODIMENTS

In the drawings, reference numeral 10 generally designates an intervertebral disc implant in accordance with various embodiments of the invention. The implant 10 comprises an envelope 12 in which a filler material 14 is received. The implant 10 is intended for use in replacing a nucleus pulposus of an intervertebral disc 16 arranged between adjacent vertebrae 18, 20. Generally, the procedure is formed in a minimally invasive manner as will be described in greater detail below.

It will be appreciated that the disc 16 comprises an annulus 22 circumscribing a nucleus pulposus. The intervertebral disc implant 10 is intended to replace a degenerate nucleus pulposus of the disc 16. Thus, the implant 10 is implanted after the disc 16 has undergone a nucleotomy to remove the nucleus pulposus.

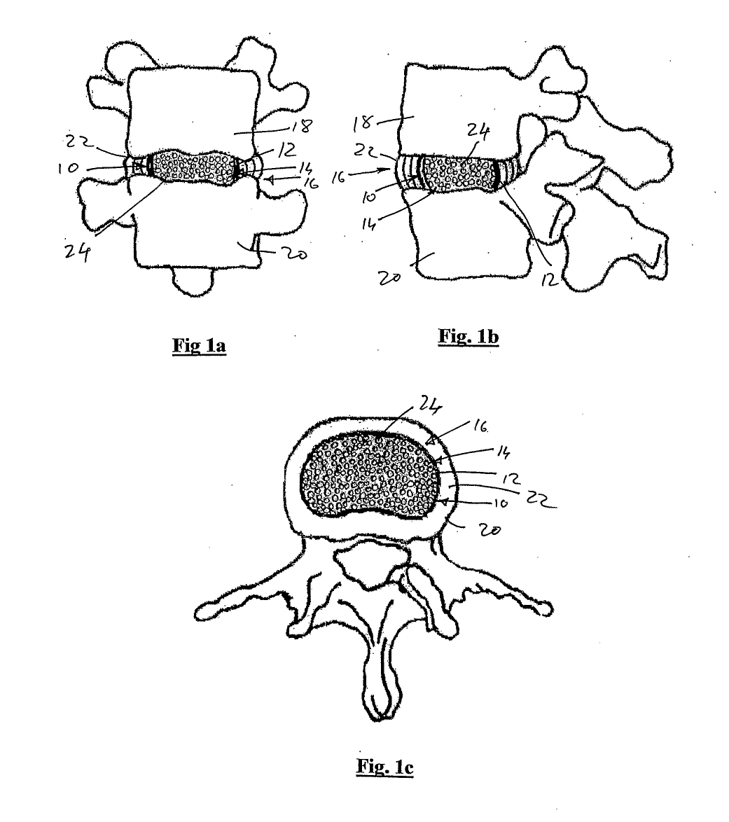

In the embodiments illustrated in FIGS. 1 to 11 of the drawings, the envelope 12 of the implant 10 is of a stretchable and elastically deformable elastomeric material such as a silicone material. Various filler materials 16 can be used with the envelope 12 in order to mimic the biomechanical actions of a natural, healthy nucleus pulposus.

In the embodiment shown in FIGS. 1a, 1b and 1c of the drawings, the filler material 14 comprises beads 24 held in suspension in a curable elastomeric material 26. The elastomeric material 26 is, once again, preferably a silicone material.

The beads 24 are of a biocompatible material. Thus, for example, the beads 24 could be of a suitable biocompatible plastics material, a biocompatible metal material, a biocompatible ceramic material or suitable biological material such as proteoglycans. The beads 24 may be homogenous in the sense that all the beads are of the same size and same material. Instead, the beads 24 may be of different sizes and different materials in order to obtain particular biomechanical characteristics for the implant 10.

The beads need not be spherical in shape. They could, instead, be any one of bullet shaped, polygonal, triangular, heart shaped, kidney shaped, ovoid, oblong, crescent shaped, cubic, elongated, conical, trapezoidal, prismatic or irregular. A preferred shape is one which allows for convenient and unobstructed insertion. Thus, preferably, the beads 24 have radiussed corners and/or edges to minimise the risk of damaging the envelope 12.

The beads may range in size from 0.01 mm to 5 mm and, optimally, any size below 4 mm in order that the beads 24 can be introduced into the interior of the envelope 12 by an introducer, as will be described in greater detail below.

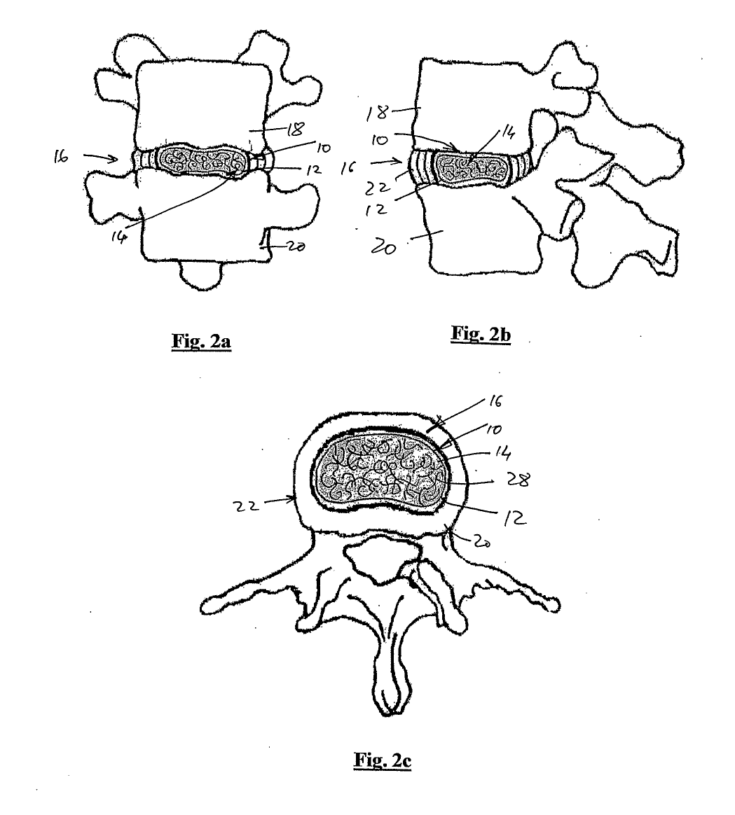

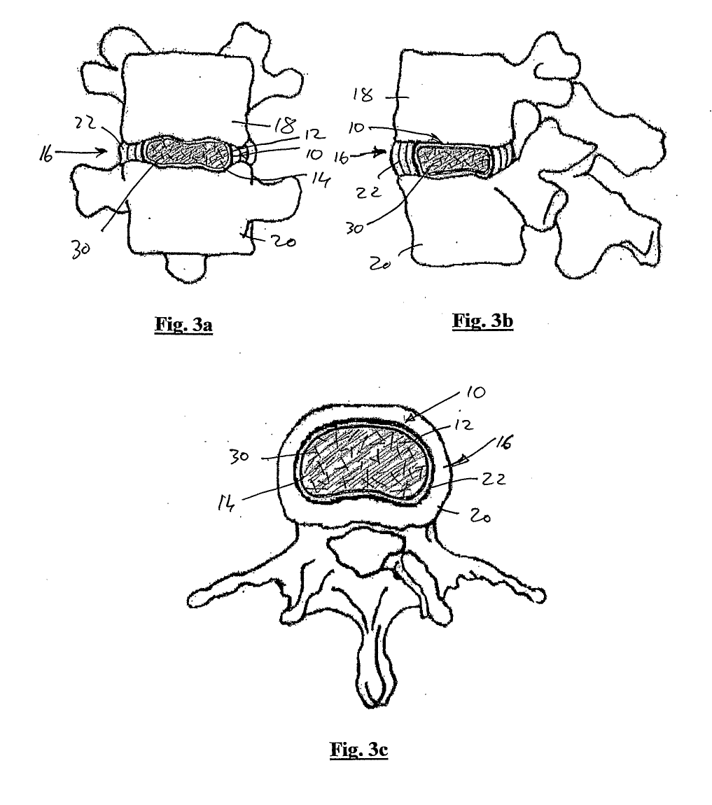

In the embodiment shown in FIGS. 2a, 2b and 2c of the drawings, the filler material 14 comprises elongate, filamentary elements carried in suspension in the silicone 26. The filamentary elements are “string-like” elements which are, once again, of suitable biocompatible materials. The elements typically have lengths not exceeding 1 cm. Once again, the lengths of the filamentary elements 26 may all be the same or they may differ to obtain the desired biomechanical characteristics for the implant 10.

In FIGS. 3a, 3b and 3c of the drawings, the filler material 14 comprises fibres 30 in suspension in the silicone 26. The fibres 30 are, typically, of lengths less than 3 mm. As in the case of the previous embodiments, the fibres are, once again, of suitable biocompatible materials. The fibres 30 are selected either to all be of the same material and lengths or they may be of different materials and of different lengths to obtain the desired biomechanical characteristics for the implant 10.

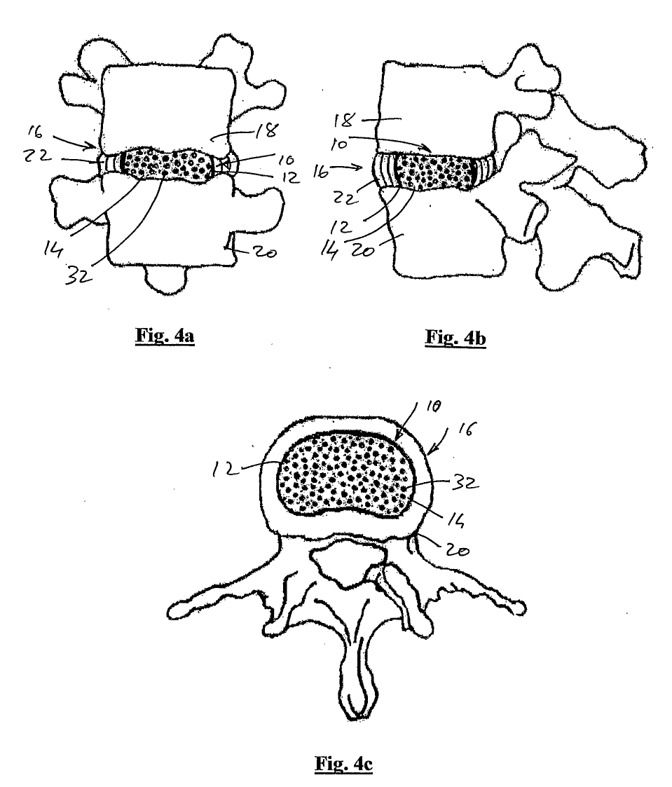

FIGS. 4a, 4b and 4c show an embodiment in which the filler material 16 comprises spherical elements contained in the envelope 12. The spherical elements 32 are of suitable biocompatible materials such as biocompatible plastics, biocompatible metals, biocompatible ceramics or biological material. The spherical objects may be in a range of sizes not exceeding 3.5 mm to 4 mm in order to be able to be introduced into the interior of the envelope 12 via an introducer as will be described in greater detail below.

The spherical elements 32 are typically provided in a range of sizes to provide compacted packing of the filler material 14 within the interior of the envelope 12 but still allowing compressive stresses on the disc 16 to be transmitted to the annulus 22 of the disc.

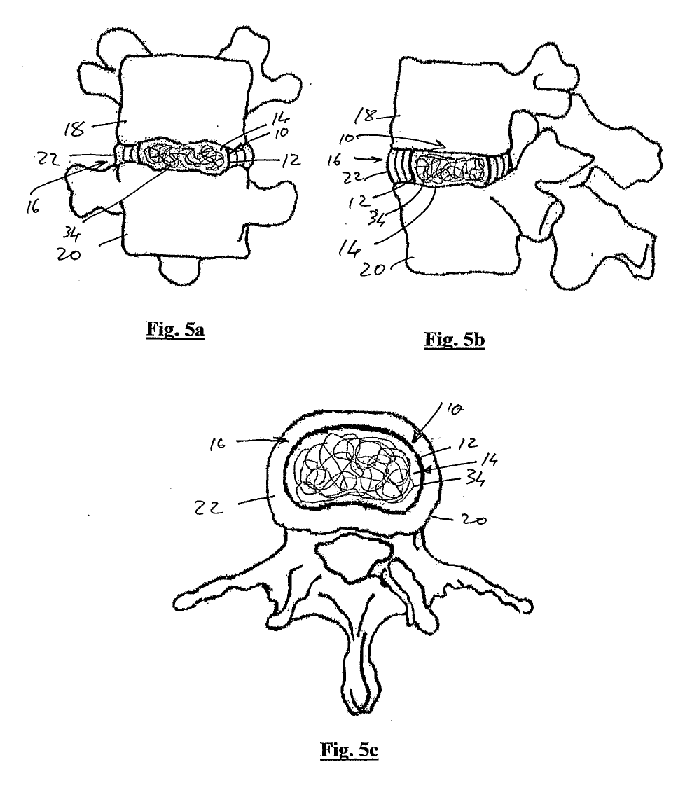

In FIGS. 5a, 5b and 5c of the drawings, the filler material 14 comprises one or more lengths of string-like elements 34. Each element 34 may typically have a length less than 10 cm and a diameter less than 3.5 mm to 4 mm. Sufficient lengths of elements 34 are provided to pack the interior of the envelope 12 to provide the necessary weight bearing functions of the implant 10. These elements 34 are, once again, of biocompatible material.

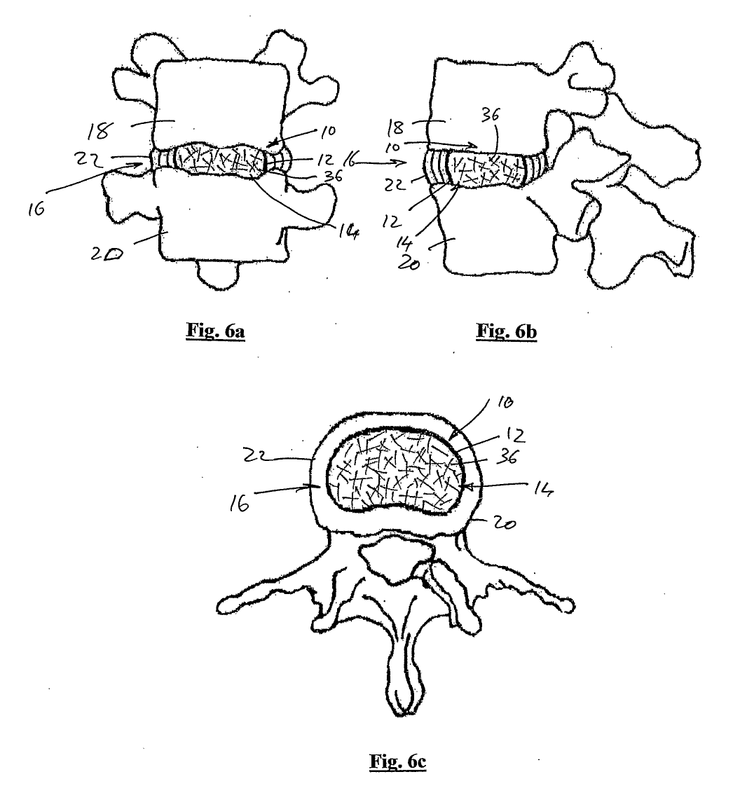

Referring now to FIGS. 6a, 6b and 6c of the drawings, the filler material 14 comprises a plurality of short lengths of fibre 36. The fibres 36 are, typically, about 2 to 3 mm long and are of biocompatible materials. The fibres 36 are packed into the interior of the envelope in a compacted state in order to impart the required biomechanical characteristics to the implant 10. Once again, the fibres 36 may be of different materials and different lengths.

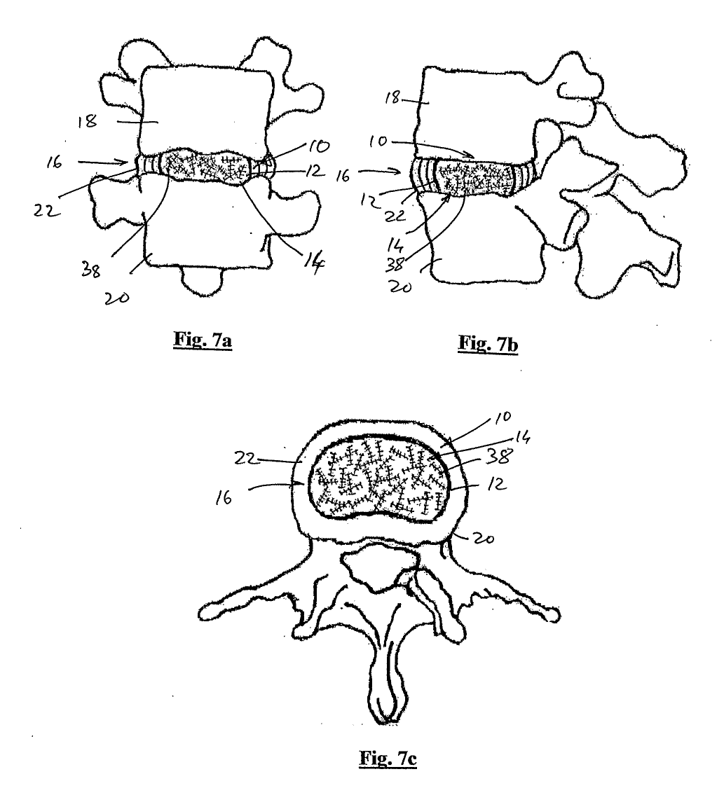

In FIGS. 7a, 7b and 7c of the drawings, the filler material 14 comprises a plurality of bottlebrush-like elements 38. The bottlebrush elements 38 are of the form having a central spine with bristles projecting radially outwardly from the spine. The bristles are folded on to the spine for introduction into the envelope 12 via an introducer.

Once again, the bottlebrush elements 38 are packed, in a compacted state, within an interior of the envelope 12 to impart the necessary biomechanical characteristics to the implant 10. The bottlebrush elements 38 can be of biocompatible plastics materials. Instead, they may be in the form of biocompatible metals/biocompatible plastics combinations. An example of this would be a bottlebrush element 38 having a metal spine with plastics bristles. Still further, the bottlebrush elements 38 could be of all metal construction. The elements 38 typically have a length of less than about 1 cm, preferably about 5 mm. When the bristles are folded on to the spine for insertion into the introducer, the element 38 may have a diameter not greater than about 3.5 mm to 4 mm.

Once again, if desired, bottlebrush elements of mixed sizes and materials may be used together to impart the desired biomechanical characteristics to the implant 10.

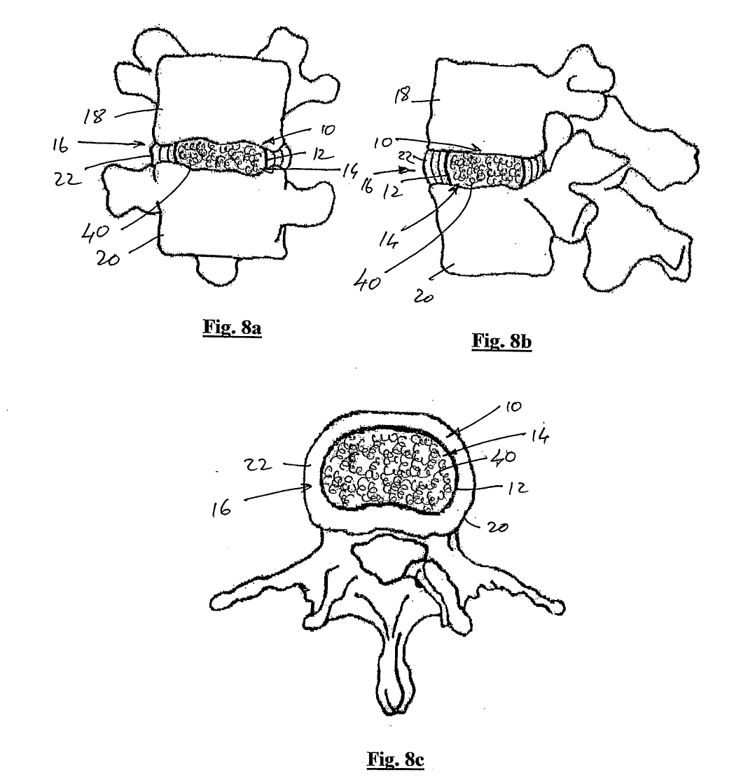

Referring now to FIGS. 8a, 8b and 8c of the drawings, the filler material 14 comprises lengths of helical or coiled wires 40. The coiled wires 40 are packed, to be in a compacted state, in the interior of the envelope 12 in order to provide the necessary biomechanical characteristics. The coiled wires, in their relaxed state, may typically be less than about 1 cm in length, preferably, about 5 mm in length. The wires 40 may be of biocompatible plastics or biocompatible metals. As in the previous embodiments, wires 40 of different lengths and different materials may be used together, if desired, in the implant 10.

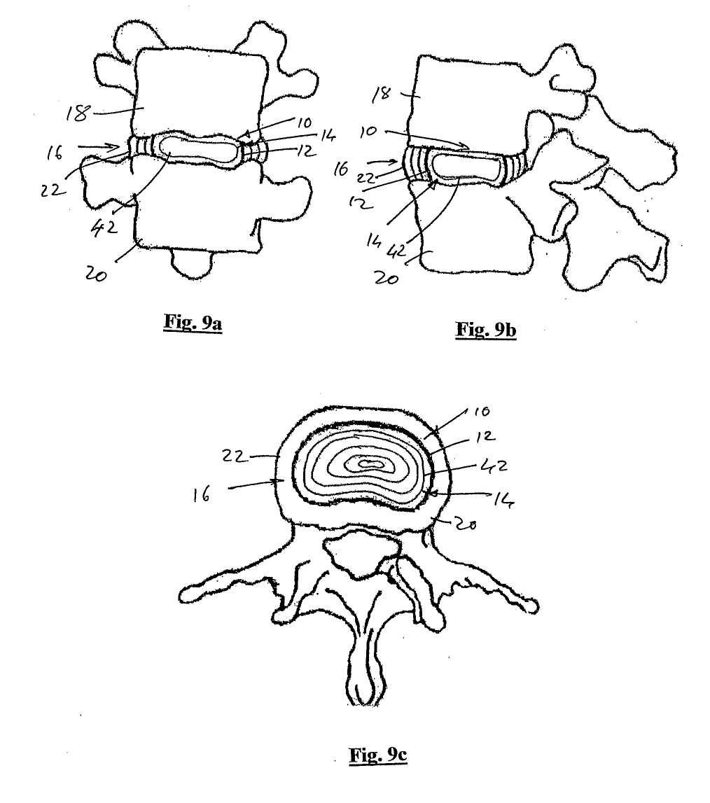

In the embodiment shown in FIGS. 9a, 9b and 9c, the filler material 14 comprises a plurality of discrete bands 42 of a resiliently flexible, biocompatible material arranged concentrically within the envelope 12 to form the implant 10. The bands 42 have a thickness not exceeding about 1 mm and a height not exceeding of about 9 mm.

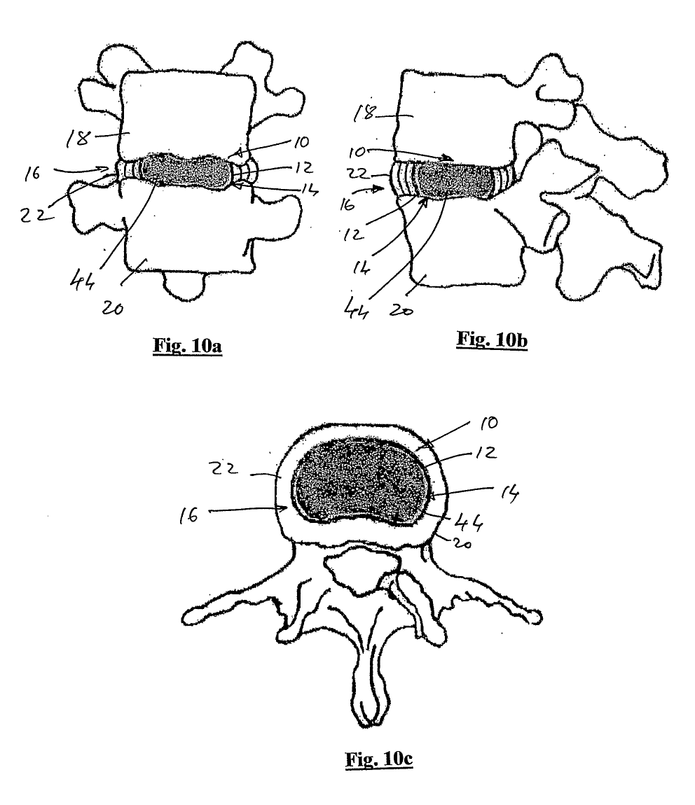

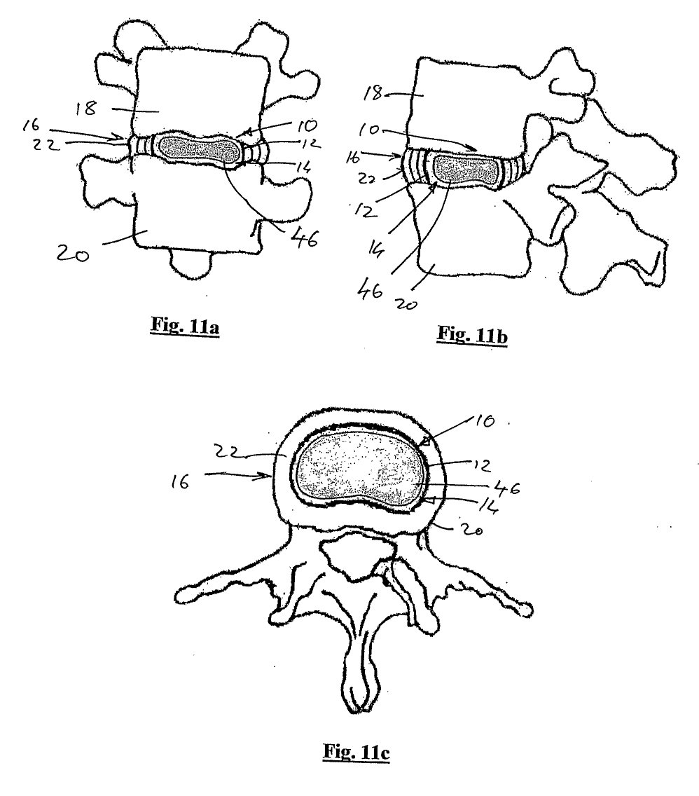

The filler material 14 in the embodiment shown in FIGS. 10a, 10b and 10c of the drawings is a foamed material 44. The foamed material 44 is introduced, in a compressed state, via the introducer into the interior of the envelope 12. Once the introducer is withdrawn, the foamed material 44 expands to a relaxed state to cause the envelope 12 to conform to the volume in which it is placed. Typically, the foamed material 44 is a polymeric material such as a polyethylene.

In FIGS. 11a, 11b and 11c of the drawings, the filler material 14 is silicone oil having a viscosity of at least 500, 000 cP. This material exhibits surprisingly good biomechanical characteristics and mimics closely a natural, healthy nucleus pulposus of an intervertebral disc.

In the embodiments described above, as previously described, the envelope 12 is generally of a silicone material which has an elongation of up to 1,000% where it can expand elastically without plastically deforming. In certain circumstances, it may not be necessary to have the envelope have such extensive elongation and, if desired, the envelope could be made of other materials in appropriate circumstances, such as, for example, woven metal fibres such as stainless steel, nitinol, chrome cobalt, titanium, or the like. Instead, the envelope may be of a plastics material such as a polymeric material like polytetrafluoroethylene.

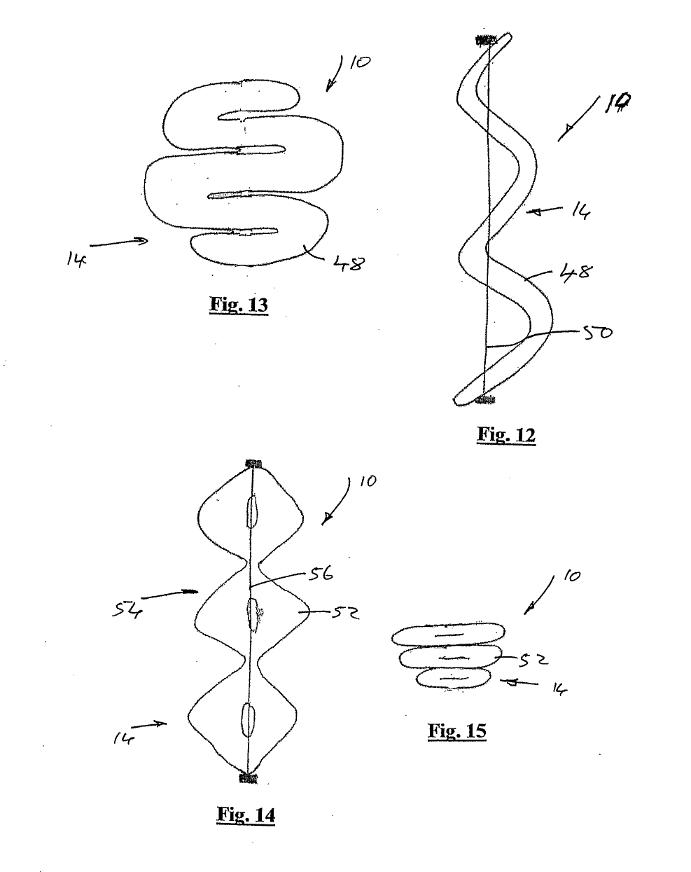

Further, in the embodiments described above, the implant 10 makes use of an envelope. In certain circumstances, the implant 10 may not require an envelope 12. In the embodiment illustrated in FIGS. 12 and 13 of the drawings, the insert 10 comprises an elongate element 48 of a suitable resiliently flexible material, such as a silicone material. In this embodiment, the element 48 is inserted into the volume resulting after the nucleotomy has been performed on the disc 16 in an elongated state as shown in FIG. 12 of the drawings. Use of a stylet 50 maintains the elongate element in its extended state. When the elongate element 48 is inserted into the volume, the stylet 50 is withdrawn causing the elongate element 48 to adopt the configuration shown in FIG. 13 of the drawings in which the element 48 substantially fills the volume. In a similar embodiment to this, a plurality of such elements 48 are used, either side by side or one on top of the other in layers, to conform to the volume. In the latter case, the elements 48 may, if desired, be inserted into a envelope (not shown).

FIGS. 14 and 15 show a similar embodiment of implant 10 in which the implant 10 comprises a plurality of doughnut-like members 52 interconnected serially to form an implantable element 54. Once again, the implantable element 54 has a stylet 56 associated with it to aid implantation.

In a relaxed state, the implantable element 54 adopts the configuration shown in FIG. 15 of the drawings. The implantable element 54 is implanted, in its first configuration, as shown in FIG. 14 of the drawings, into the volume of the disc 16. Withdrawal of the stylet 56 causes the implantable element 54 to be compressed, as shown in FIG. 15 of the drawings, into a second configuration in which it conforms substantially to the volume of the disc 16.

Once again, in a similar manner to the embodiment described above with reference to FIGS. 12 and 13 of the drawings, a plurality of the implantable elements 54 may be used, either side by side or in layers to conform to the volume of the disc 16. In this case, the implantable elements 54 may be received in an envelope (not shown).

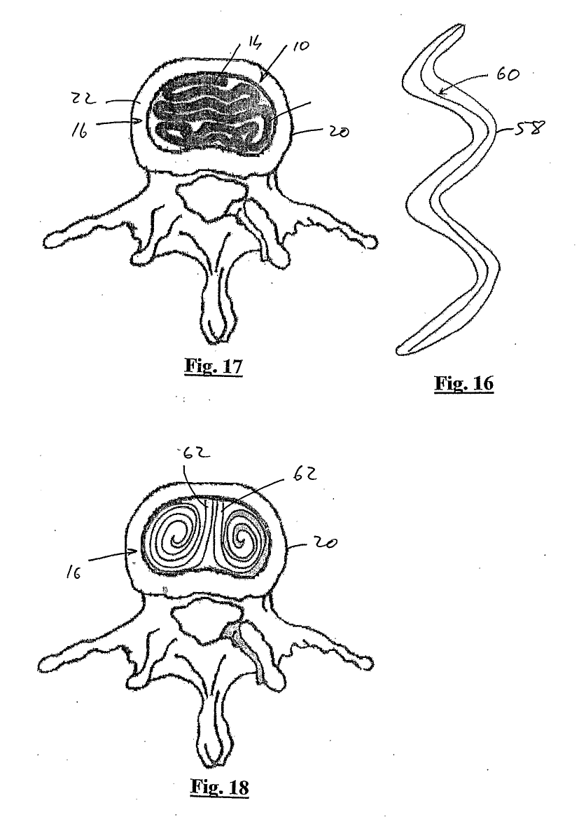

Referring now to FIGS. 16 and 17 of the drawings, yet a further embodiment of an implant 10 is illustrated.

In this embodiment, the implant 10 comprises an elongate implantable element 58 which, optionally, has a stiffening spine 60. The implantable element 58 is, typically, an elastomeric material such as, for example, silicone. The spine 60 is of a shape forming material or shape memory alloy such as nitinol.

The implantable element 58 is inserted via an introducer into the volume of the disc 16. One or more lengths of the implantable elements 58 may be used to cause the implantable elements 58 to conform to the shape of the volume in order to function as a replacement nucleus pulposus of the disc 16.

In FIG. 18 of the drawings, an embodiment similar to that described above with reference to FIGS. 16 and 17 is illustrated. In this embodiment, the implant 10 comprises two, coiled implantable elements. Each implantable element 62 has a coiled shaped in its relaxed state. This coiled shape may be imparted by a stiffening spine of shape forming alloy such as nitinol (not shown). Instead, the implantable elements 62 may be formed in such a manner that, in their relaxed state, they adopt a coiled configuration.

In this embodiment, the implantable elements 62 are straightened for introduction into the volume of the disc 16. Once in the volume, the implantable elements 62 coil in oppositely directed orientations substantially to fill the volume resulting from removal of the original nucleus pulposus of the disc 16.

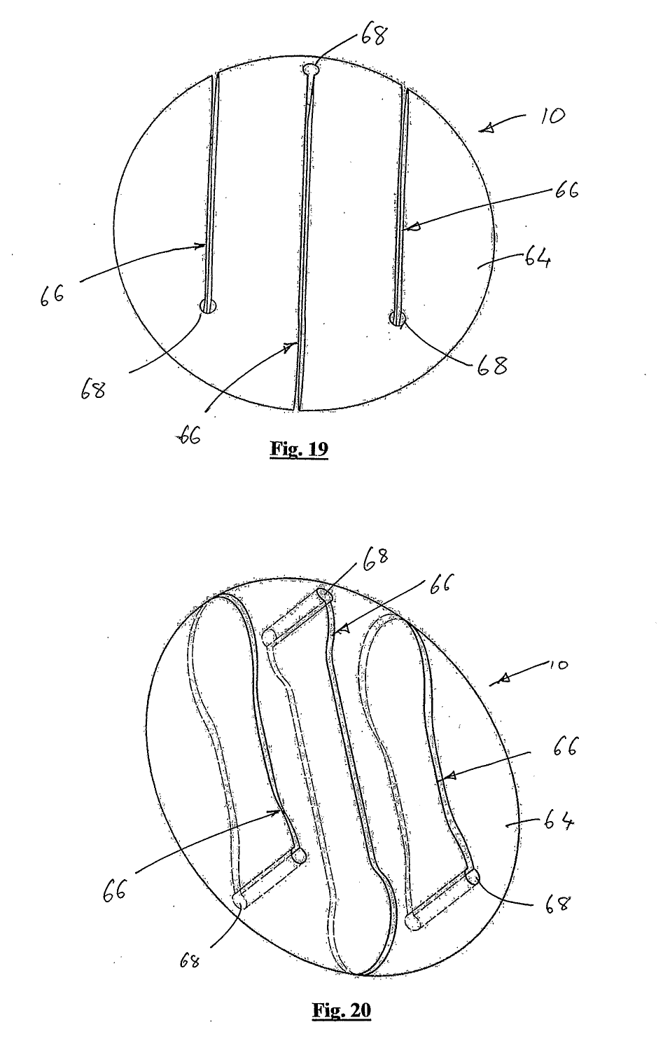

In FIGS. 19 and 20 of the drawings, the implant 10 comprises a single, implantable element 64. The implantable element 64 is of an elastomeric material, such as silicone, and, in its relaxed states, is in a shape which will substantially conform to the volume of the disc into which the element 64 is to be imparted.

To aid in implantation of the element 64, a plurality of cuts 66 are made in the element. These cuts 66 cause “hinges” 68 to be formed about which the parts of the element on either side of the cut 66 can hinge to straighten the element 64 to be implanted via an introducer into the vacated volume of the disc 16.

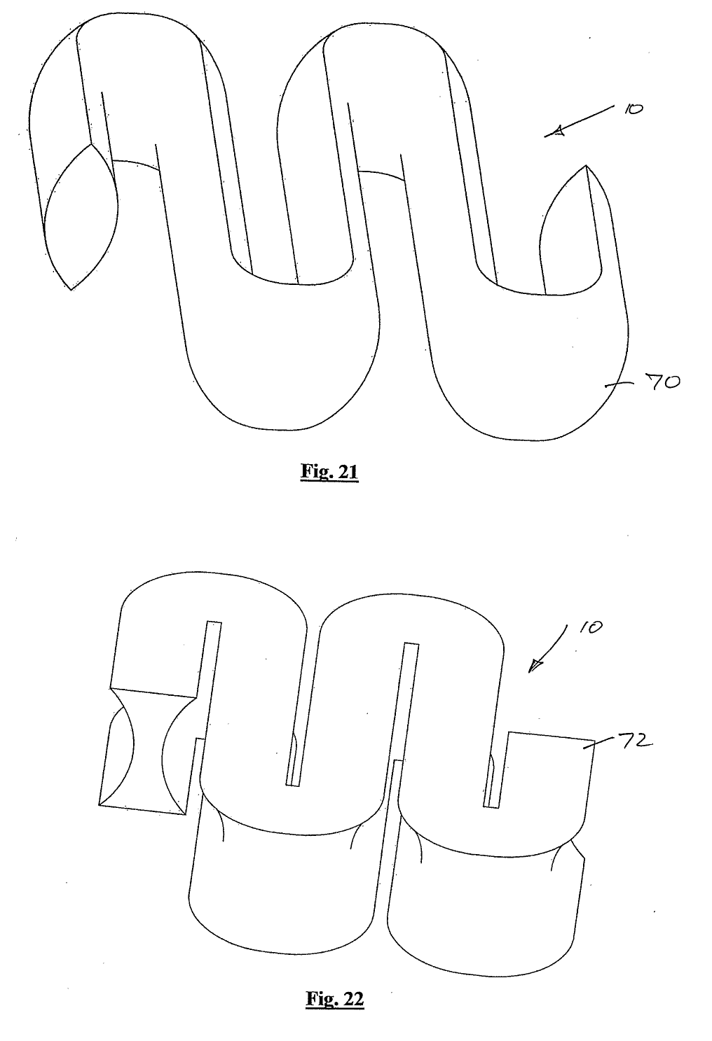

The embodiments of the implants shown in FIGS. 21 and 22 of the drawings are similar to those shown in FIGS. 19 and 20 of the drawings. In the embodiment shown in FIG. 21 of the drawings, the implant 10 comprises a single implantable element 70 formed into a snake-like configuration, in its relaxed state. The implantable element 70 has a convex profile. The embodiment shown in FIG. 22 of the drawings is of a similar form with the distinction that an implantable element 72 of the implant 10 of the embodiment shown in FIG. 22 has a concave profile. Once again, in both embodiments, the implantable element 70, 72 is extended into a straight configuration for implantation via an introducer. Once in the volume of the disc 16, the implantable element 70, 72 adopts its relaxed, illustrated configuration substantially to conform to the volume of the disc 16.

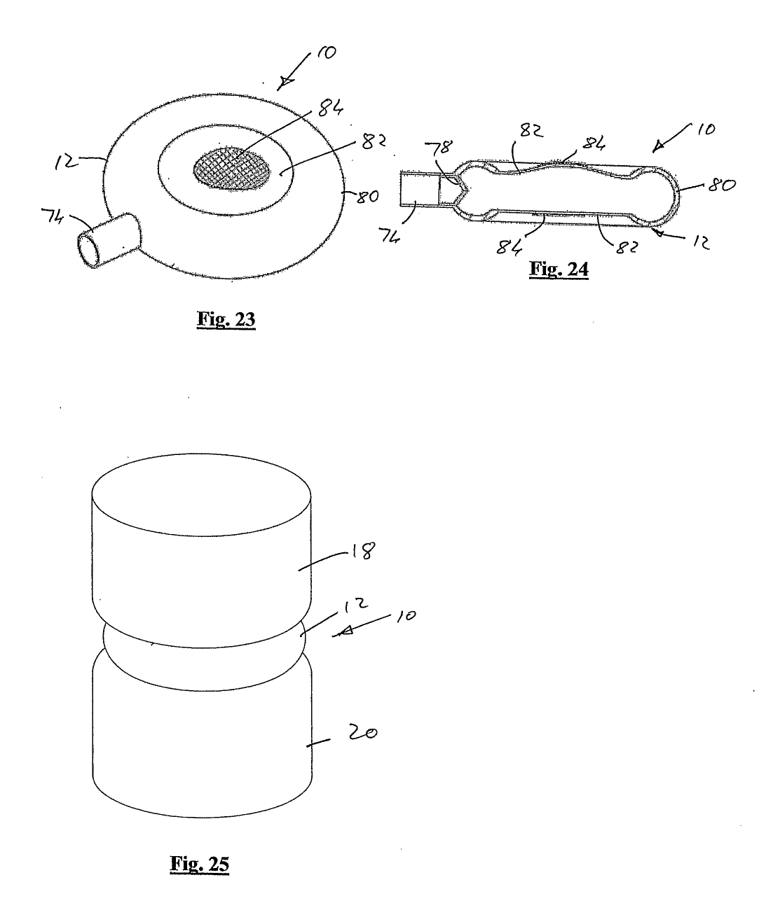

Yet a further embodiment of an implant 10 is shown in FIGS. 23 to 25 of the drawings. Once again, with reference to the previous embodiments, like reference numerals refer to like parts, unless otherwise specified.

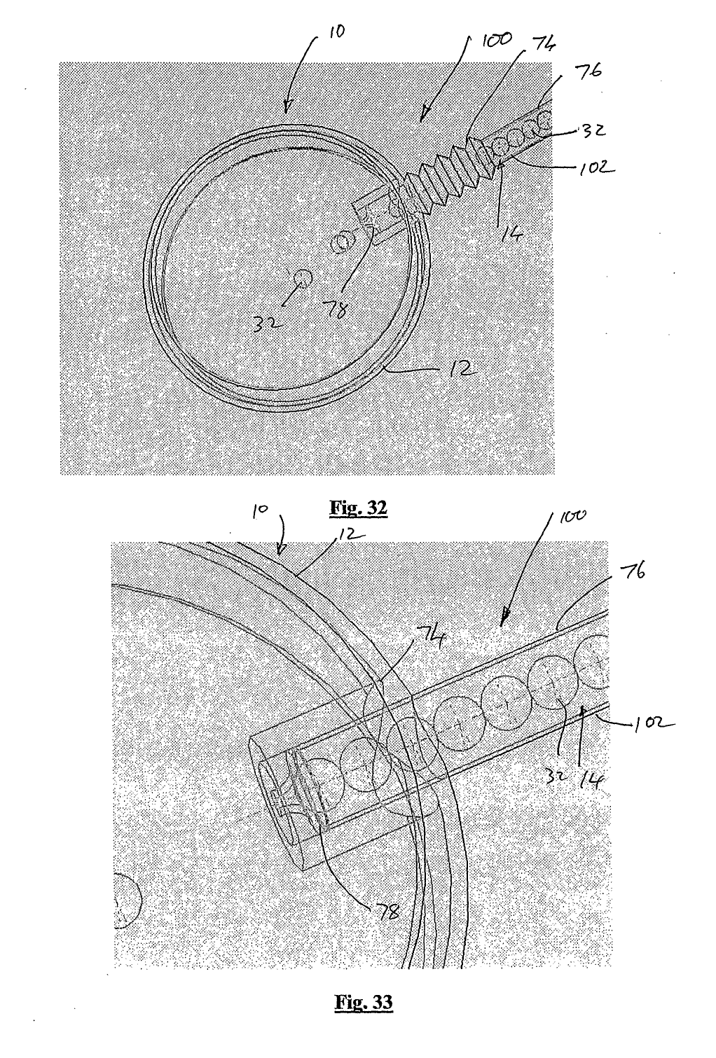

In this embodiment, an attaching formation 74 of the envelope 12 is clearly shown. It is to be understood that the envelope 12 of each of the embodiments described above also includes such an attaching formation. The attaching formation 74 is used for attaching the envelope to an introducer 76 (FIG. 32). The attaching formation 74 is in the form of a filler tube. The filler tube 74, in this embodiment, extends radially outwardly from the body of the envelope 12. A closure device in the form of a duck-billed valve 78 is arranged at a distal end of the filler tube 74. When the introducer 76 is inserted into the filler tube 74, it causes the valve 78 to open. Withdrawal of the introducer 76 from the filler tube 74 causes the valve 78 to close.

In this embodiment of the invention, the envelope 12 has an annular region 80 of a reasonably rigid material. The material of the annular region 80 is more rigid than material forming upper and lower members 82 of a central part of the envelope 12. The annular region 80 of the envelope 12 bears against the annulus 22 of the disc 16, in use. When the filler material 14 is charged into the interior of the envelope 12, the members 82 expand outwardly as shown by the upper member 82 in FIG. 24 of the drawings to bear against the vertebrae 18, 20 and so cause the envelope 12 to conform substantially to the volume of the disc 16.

It is to be noted that both members 82 carry, on their outer surfaces, a layer of tissue ingrowth material 84. The material 84 is, typically, a polyester material such as that sold under the registered trade mark Dacron.

The annular region 80 is of a substantially non-stretchable material while the members 82 are made to stretch and expand in volume. The material of the annular region 80 is still sufficiently flexible to enable the envelope 12 to be collapsed to be inserted via an introducer into the vacated volume of the disc 16.

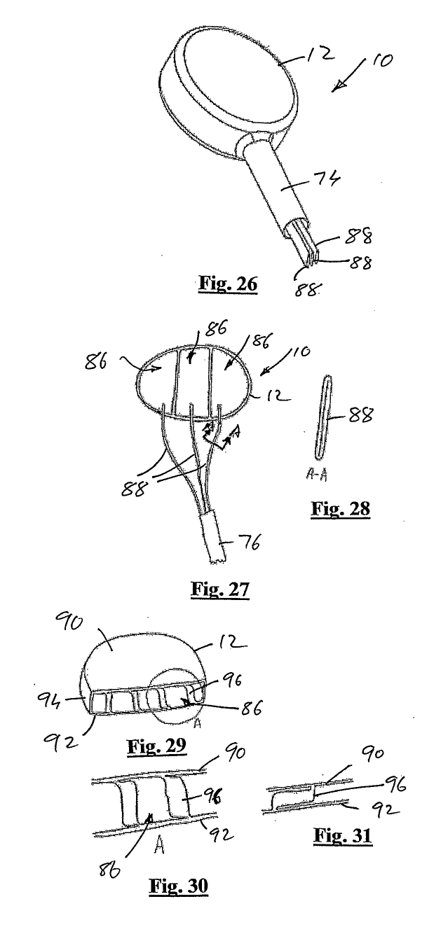

FIGS. 26 to 31 show various embodiments of a multi-chambered envelope 12. As shown most clearly in FIG. 27 of the drawings, the envelope 12 has a plurality of chambers 86, each of which is fed by a collapsible delivery tube 88. Each delivery tube 88 has a valve (not shown) at its distal end. Filler material is introduced into each of the chambers 86 of the envelope 12 via the associated delivery tube 88. Thus, filling of each of the chambers 86 can occur independently. In addition, the filler material received in each chamber 86 may differ from the filler material received in any other chamber 86. Still further, certain of the chambers 86 may, in certain circumstances, not have any filler material at all.

A sample of the construction of the envelope 12 is shown in FIGS. 29 to 31 of the drawings. The envelope 12 has an upper member 90 and a lower member 92 interconnected by a sidewall 94. A plurality of partitions 96 extend in the interior of the envelope 12 between the upper member 90 and the lower member 92. The partitions 96 are configured to have strong compressive load bearing capabilities but to collapse in shear as shown in FIG. 31 of the drawings. Thus, for introduction of the envelope into the vacated volume of the disc 16, the partitions 96 are collapsed, as shown in FIG. 31 of the drawings by moving the members 90 and 92 laterally relative to each other.

It will be appreciated that various other configurations of multi-chambered envelopes 12 can be formed by using different materials for different chambers of the envelope and/or filling the various chambers with different filler materials 14, as described above.

In FIGS. 32 and 33 of the drawings, a system, in accordance with another embodiment of the invention, for implanting an intervertebral disc implant is shown and is illustrated generally by the reference numeral 100. The system 10 comprises the implant 10 and an introducer 76. The introducer 76 has an elongate tubular element 102 on a distal end of which is received the attaching formation 74 of the envelope 12. A non-return valve 78 is arranged at a distal end of the attaching formation 74. In the embodiment illustrated in FIGS. 32 and 33 of the drawings, the filler material comprises the balls 32 of the embodiment described above with reference to FIGS. 4a, 4b and 4c of the drawings.

The annulus 22 of the disc 16 is accessed percutaneously in a patient and an opening is made through the annulus 22. The degenerate nucleus pulposus is removed using ablation, lasers or mechanical means to create a vacated volume. The introducer 76 with the envelope 12 in a collapsed configuration on the distal end of the tubular member 102 is inserted through the incision so that the envelope 12 is within the volume of the disc 16.

Filler material 14 is fed through the tubular member 102 of the introducer 76 into the interior of the envelope 12 to cause the envelope 12 to expand to conform to the volume of the disc 16. In the embodiment shown in FIGS. 32 and 33 of the drawings, the filler material is fed through the introducer via an appropriate displacement mechanism, such as a pump (not shown). Once the envelope 12 has expanded to conform to the volume, charging of filler material 14 into the interior of the envelope 12 ceases. The tubular member 102 of the introducer 76 is withdrawn from the attaching formation 74 of the envelope 12. Withdrawal of the tubular member 102 causes the value 78 to close inhibiting leakage of the filler material 14 from within the envelope 12.

It will be appreciated that the balls 32 have been shown merely as one example of the type of filler material 14 used with the introducer 76. Other filler materials 14 having discrete elements are also able to be injected into the envelope 12 of the implant 10 using the introducer 76.

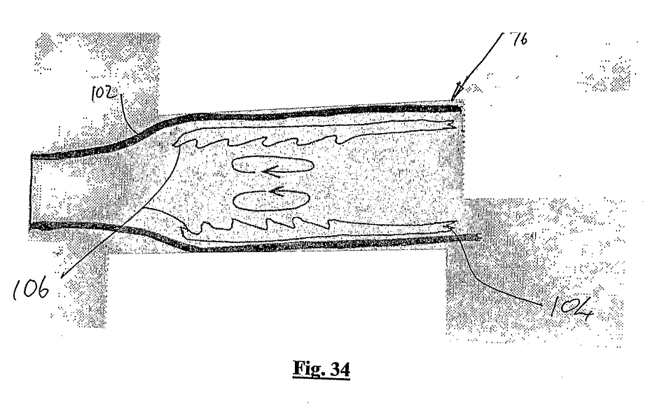

In FIG. 34 of the drawings, part of another embodiment of an introducer is illustrated. With reference to the previous embodiment, like reference numerals refer to like parts, unless otherwise specified. In this embodiment of the invention, the displacement mechanism for charging filler material 14 into the interior of the envelope 12 comprises a displaceable element 104. The displaceable element 104 is a sleeve received within the tubular member 102 of the introducer 76 and which is able to reciprocate relative to the tubular member 102. An inner surface of the sleeve 104 carries a ratchet arrangement 106. By reciprocating the sleeve 104 relative to the tubular member 102 filler material 14 can be fed along the introducer 76 into the interior of the envelope 12 by means of the ratchet arrangement. The introducer 76 of the embodiment shown in FIG. 34 of the drawings is useful for introducing elongate elements into the interior of the envelope or, in certain circumstances, such as the embodiments shown in FIGS. 12 to 22 directly into the volume where no envelope is used. An example of an implant 10 which would use the introducer 76 of the embodiment of FIG. 34 is that shown in FIGS. 5a, 5b and 5c of the drawings as well as the embodiment shown in FIGS. 9a, 9b and 9c of the drawings.

It is to be noted that the implant 10 may be used to deliver bioactive substances to the annulus 22 of the intervertebral disc 16. The bioactive substances may be substances which induce cell growth and/or cell reproduction. Further, the implant 10 may be used as a drug delivery means for active and/or prophylactic treatment at the site of implantation. Substances to be delivered may include may include gene telomerase, proteins, cells, autologous chondrocytes and autologous bone marrow derived mesenchymal stem cells.

Hence, it is an advantage of the invention, that an intervertebral disc implant is provided which can mimic the biomechanical characteristics of a natural, healthy nucleus fibrosis of an intervertebral disc. It is a particular advantage of the invention that an implant and system are provided which enables the implant to be inserted in a minimally invasive manner thereby obviating the need for drastic surgery. By use of discrete elements for the filler material 14, the biomechanical properties of the implant 10 can be tailored to particular requirements as desired by a clinician.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An intervertebral disc implant which includes:
an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and
a filler material receivable in the envelope via the introducer to cause the envelope to expand elastically to conform substantially to the volume in which the envelope is received, in use.

2. The implant of claim 1 in which the envelope is of a silicone material.

3. The implant of claim 1 in which the attaching formation comprises a filler tube mountable to the introducer, the attaching formation including a closure device to inhibit back flow of filler material.

4. The implant of claims 1 in which the filler material comprises a plurality of discrete, biocompatible elements.

5. The implant of claim 4 in which the elements includes, singly or in combination, beads, elongate elements and expansible elements.

6. The implant of claim 5 in which the elongate elements are selected from the group consisting of fibres, lengths of filamentary elements, bristle carrying elements and helical elements.

7. The implant of claim 4 in which the discrete elements are arranged in suspension in a filler within the volume.

8. The implant of claim 7 in which the filler is an elastomeric, curable filler.

9. The implant of claim 5 in which each expansible element is configured to change from a first configuration for insertion into the envelope to a second configuration which causes the envelope to conform substantially to the volume.

10. The implant of claim 9 in which each expansible element is configured to be received, in its first configuration, in the introducer for introduction into the envelope.

11. The implant of claim 9 in which each expansible element, in its rest condition, adopts the second configuration.

12. The implant of claim 4 in which the filler material is a foamed material which is introduced in a compressed state via the introducer into the interior of the envelope where it expands to its relaxed state to cause the envelope to conform to the volume.

13. The implant of claim 12 in which the foamed material is a polymeric material.

14. The implant of claim 4 in which the filler material comprises a plurality of discrete bands of a resiliently flexible material.

15. The implant of claim 14 in which the bands are configured to be arranged concentrically within the envelope.

16. The implant of claim 1 in which the envelope carries at least one layer of a tissue ingrowth material.

17. An intervertebral disc implant which includes:
an envelope, the envelope including an attaching formation for attachment to an introducer to enable the envelope, in a collapsed state, to be introduced into a volume of an intervertebral disc that has undergone a nucleotomy; and
a filler material receivable in the envelope after placement of the envelope in the volume of the disc, in use, to cause expansion of the envelope to conform to the volume, the filler material comprising a plurality of discrete, elongate elements introducible, via the introducer, into an interior of the envelope.

18. The implant of claim 17 in which the envelope is of an expansible material.

19. The implant of claim 18 in which the envelope carries at least one layer of a tissue ingrowth material.

20. The implant of claim 17 in which the envelope defines a filler opening and includes a closure element for closing the opening after introduction of the filler material.

21. The implant of claim 17 in which the elongate elements are selected from the group consisting of fibres, lengths of filamentary elements, bristle carrying elements and helical elements.

22. The implant of claim 17 in which the elongate elements are arranged in suspension in a filler within the volume.

23. The implant of claim 22 in which the filler is an elastomeric, curable filler.

24. The implant of claim 17 in which the elongate elements comprise a plurality of discrete bands of a resiliently flexible material.

25. The implant of claim 24 in which the bands are configured to be arranged concentrically within the envelope.

26. The implant of claim 17 in which the elongate elements are expansible elements.

27. The implant of claim 26 in which each expansible element is configured to change from a first configuration for insertion into the envelope to a second configuration which causes the envelope to conform substantially to the volume.

28. The implant of claim 27 in which each expansible element is configured to be received, in its first configuration, in the introducer for introduction into the envelope.

29. The implant of claim 25 in which each expansible element, in its rest condition, adopts the second configuration.

30. An intervertebral disc implant which includes:
an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and
a filler material receivable in the envelope via the introducer to cause the envelope to expand elastically to conform substantially to the volume in which the envelope is received, in use, the filler material being a foamed material which is introduced in a compressed state via the introducer into the interior of the envelope where it expands to its relaxed state to cause the envelope to conform to-the volume.

31. The implant of claim 30 in which the foamed material is a polymeric material.

32. The implant of claim 30 in which the envelope carries at least one layer of a tissue ingrowth material.

33. The implant of claim 30 in which the envelope defines a filler opening and includes a closure element for closing the opening after introduction of the packing material.

34. An intervertebral disc implant which includes:
an envelope, the envelope including an attaching formation for attachment to an introducer to enable the envelope, in a collapsed state, to be introduced, in a minimally invasive manner, into a volume of an intervertebral disc that has undergone a nucleotomy; and
a filler material receivable in the envelope after placement of the envelope in the volume of the disc, in use, to cause expansion of the envelope to conform to the volume, the filler material comprising, in combination, a curable filler material and a plurality of discrete, biocompatible elements contained, in use, in the filler material within the envelope.

35. The implant of claim 34 in which the envelope is of an expansible material.

36. The implant of claim 35 in which the envelope carries at least one layer of a tissue ingrowth material.

37. The implant of claim 34 in which the envelope defines a filler opening and includes a closure element for closing the opening after introduction of the packing material.

38. The implant of claim 34 in which the elements includes, singly or in combination, beads, elongate elements and expansible elements.

39. The implant of claim 38 in which the elongate elements are selected from the group consisting of fibres, lengths of filamentary elements, bristle carrying elements and helical elements.

40. The implant of claim 38 in which each expansible element is configured to change from a first configuration for insertion into the envelope to a second configuration which causes the envelope to conform substantially to the volume.

41. The implant of claim 40 in which each expansible element is configured to be received, in its first configuration, in the introducer for introduction into the envelope.

42. The implant of claim 40 in which each expansible element, in its rest condition, adopts the second configuration.

43. The implant of claim 34 in which the filler is an elastomeric, curable filler.

44. An intervertebral disc implant which includes:
an envelope of a stretchable and elastically deformable elastomeric material, the envelope including an attaching formation for attachment to an introducer to enable the envelope, in a collapsed state, to be introduced into a volume of an intervertebral disc that has undergone a nucleotomy; and
a filler material receivable in the envelope via the introducer to cause the envelope to expand elastically to conform substantially to the volume in which the envelope is received, in use, the filler material being an elastomeric material having a viscosity of at least 500000 cP.

45. The implant of claim 44 in which the elastomeric material is a silicone.

46. The implant of claim 44 in which the envelope carries at least one layer of a tissue ingrowth material.

47. The implant of claim 44 in which the envelope defines a filler opening and includes a closure element for closing the opening after introduction of the packing material.

48. An intervertebral disc implant which includes:
an envelope receivable in a volume of an intervertebral disc that has undergone a nucleotomy, the envelope defining a plurality of chambers, the chambers being configured so that, when at least certain of the chambers contain a filler material, the envelope conforms substantially to the volume of the disc;
a filler material receivable in the at least certain of the chambers; and
at least one of the chambers having a filler mechanism associated with it.

49. The implant of claim 48 in which the chambers are defined by wall portions of the envelope, wall portions of some of the chambers being of a different wall thickness than wall portions of other chambers.

50. The implant of claim 48 in which the chambers are defined by wall portions of the envelope, wall portions of some of the chambers being of a different material than wall portions of other chambers.

51. The implant of claim 47 in which the filler material receivable in at least one of the chambers differs from the filler material that is receivable in at least one other of the chambers.

52. The implant of claim 47 in which the envelope includes an attaching formation for attachment to a tubular introducer to enable the envelope, in a collapsed state, to be introduced into the volume of the disc.

53. The implant of claim 52 in which each chamber in which filler material is receivable has a filler mechanism associated with it.

54. The implant of claim 53 in which the filler mechanism is a one-way device that, upon closure, inhibits back flow of filler material.

55. The implant of claim 54 in which the filler mechanism of an outer chamber of the envelope is implemented as the attaching formation.

56. The implant of claim 47 in which the envelope carries at least one layer of a tissue ingrowth material.

57. A system for implanting an intervertebral disc implant, the system comprising:
an implant as claimed in claim 53; and
an introducer, the introducer comprising a plurality of filler tubes, each tube communication independently of any other tube with its associated chamber of the envelope for charging filler material into the associated chamber.

58. An intervertebral disc implant which includes at least one element which changes from a first configuration for insertion into a volume of an intervertebral disc that has undergone a nucleotomy to a second configuration in which the at least one element conforms substantially to the volume, the at least one element being configured to be received, in its first configuration, in an introducer to be inserted into the volume of the disc.

59. The implant of claim 58 in which the at least one element, in its first configuration, is elongate and, in its second configuration, adopts a shape conforming substantially to the volume.

60. The implant of claim 58 in which the at least one element includes a biocompatible, shape memory alloy which causes the element to adopt its second configuration in the volume after ejection from the introducer.

61. The implant of claim 60 in which the at least one element, in its relaxed state, is in the first configuration, the at least one element including a retention device for retaining the at least one element in the second configuration after ejection from the introducer.

62. The implant of claim 58 which includes:
an envelope receivable in a collapsed state in the volume; and
a plurality of the elements receivable in the envelope, the plurality of elements causing the envelope to expand substantially to conform to the volume.

63. A system for implanting an intervertebral disc implant as claimed in any one of the preceding claims, the system including:
an introducer having a proximal and a distal end, a mount for the envelope of the implant being arranged at or adjacent the distal end of the introducer;
a source of filler material connectable to the proximal end of the introducer; and
a displacement mechanism for displacing the filler material along the introducer to be ejected from the introducer into the envelope, in use.

64. The system of claim 63 in which the introducer comprises at least one tubular member.

65. The system of claim 64 in which the introducer comprises at least two tubular members arranged in a telescopic fashion, the tubular members being reciprocally displaceable relative to one another.

66. The system of claim 65 in which an innermost of the tubular members carries the displacement mechanism.

67. The system of claim 66 in which the displacement mechanism comprises a ratchet arrangement for urging the filler material along the introducer into the envelope.

68. A method of implanting an intervertebral disc implant into an intervertebral disc, the method including:
percutaneously performing a nucleotomy on the disc to remove a nucleus pulposus of the disc to create a volume;
inserting an envelope of the implant into the volume;
charging an interior of the envelope with filler material in a manner to allow the envelope to expand to conform substantially to the volume; and
causing the interior of the envelope to be closed off to retain the filler material within the envelope, the filler material being selected to mimic natural biomechanical characteristics of the nucleus pulposus of the disc.

69. The method of claim 68 which includes inserting the envelope into the volume using an introducer, the envelope being placed in a collapsed state on a distal end of the introducer and inserted percutaneously through an opening in an annulus of the disc.

70. The method of claim 69 which includes charging the filler material into the interior of the envelope through the introducer.

71. The method of claim 70 which includes closing off the interior of the envelope by sealing a wall of the envelope.

72. The method of claim 71 which includes closing off the interior of the envelope by the action of withdrawing the introducer from the envelope.

73. A method of implanting an intervertebral disc implant into an intervertebral disc, the method including:
percutaneously performing a nucleotomy on the disc to remove a nucleus pulposus of the disc to create a volume;
inserting an introducer into an opening formed in an annulus of the disc; and
introducing into the volume, via the introducer, at least one element which changes from a first configuration, in which the at least one element is able to be inserted into the introducer, to a second configuration in which the at least one element conforms substantially to the volume.

74. The method of claim 73 which includes using a single element which, in its second configuration, conforms substantially to the volume of the disc.

75. The method of claim 73 which includes using a plurality of elements which together, when each such element is in its second configuration, conform substantially to the volume of the disc.

76. The method of claim 75 which includes, prior to insertion of the elements in the volume, introducing an envelope, in a collapsed state, into the volume and introducing the elements into the envelope to cause the envelope to expand to conform substantially to the volume of the disc.

77. The method of claim 76 which includes, after introduction of the elements into the envelope, closing off a filler opening of the envelope.

78. The method of claim 77 which includes closing off the filler opening of the envelope by withdrawal of the introducer from the filler opening of the introducer.

79. An introducer for introducing an intervertebral disc implant into a disc that has undergone a nucleotomy, the introducer including:
at least two sleeves arranged telescopically with respect to each other; and
a displacement mechanism arranged on an operatively inner surface of an innermost one of the sleeves for assisting in displacing filler material along the sleeves into an interior of the disc, in use.

80. The introducer of claim 79 in which the displacement mechanism comprises a ratchet arrangement for urging the filler material along the sleeve.
